(19) United States
(12) Patent Application Publication
(10) Pub. No.: US 2025/0178988 A1
(43) Pub. Date: Jun. 5, 2025

(54) ANTI-CARBON DEPOSITION CATALYST, PREPARATION METHOD THEREFOR AND USE THEREOF

(71) Applicants: SINOCHEM LANTIAN CO., LTD., Hangzhou, Zhejiang Province (CN); ZHEJIANG LANTIAN ENVIROMENTAL PROTECTION HI-TECH CO., LTD., Hangzhou, Zhejiang Province (CN); ZHEJIANG RESEARCH INSTITUTE OF CHEMICAL INDUSTRY CO., LTD., Hangzhou, ZHEJIANG PROVINCE (CN)

(72) Inventors: WUCAN LIU, HANGZHOU, ZHEJIANG PROVINCE (CN); LING LI, HANGZHOU, ZHEJIANG PROVINCE (CN); CHAOFENG MA, HANGZHOU, ZHEJIANG PROVINCE (CN); CHUNSHAN LU, HANGZHOU, ZHEJIANG PROVINCE (CN); JIANJUN ZHANG, HANGZHOU, ZHEJIANG PROVINCE (CN); XIAONIAN LI, HANGZHOU, ZHEJIANG PROVINCE (CN); JIAMIN JIN, HANGZHOU, ZHEJIANG PROVINCE (CN)

(21) Appl. No.: 18/843,296

(22) PCT Filed: Mar. 3, 2023
(86) PCT No.: PCT/CN2023/079588

§ 371 Date: Sep. 2, 2024

(30) Foreign Application Priority Data
Mar. 4, 2022 (CN) 202210207172.0

(51) Int. Cl. C07C 17/23; B01J 21/18; B01J 23/60; B01J 23/89; B01J 37/02; B01J 37/06; B01J 37/08; B01J 37/18

(52) U.S. Cl. CPC C07C 17/23; B01J 21/18; B01J 23/60; B01J 23/892; B01J 23/8926; B01J 37/0207; B01J 37/0213; B01J 37/0236; B01J 37/06; B01J 37/088; B01J 37/18

(57) ABSTRACT

The present invention are an anti-carbon deposition catalyst, a preparation method therefor and use thereof. The anti-carbon deposition catalyst is composed of a carbon carrier, a metal active component, a metal auxiliary agent I, and a metal auxiliary agent II. The metal active component is platinum or palladium, the metal auxiliary agent I is zinc or copper or cobalt, the metal auxiliary agent II is ruthenium or nickel, and each type of metal component includes one and only one metal. The metal active component accounts for 0.2-2.0% of a mass content of the carrier, and a mass ratio of the metal active component, the metal auxiliary agent I and the metal auxiliary agent II is 1:(1-10):(0.01-0.001). Three metals in the anti-carbon deposition catalyst of the present invention can form a multifunctional catalytic activity center to achieve in-situ elimination of carbon deposits through hydrogenation while maintaining high dechlorination catalytic performance, thereby effectively inhibiting the generation of carbon deposits and greatly improving the stability and service life of the catalyst.

### FIELD OF TECHNOLOGY

The present invention relates to the field of catalysis, in particular to an anti-carbon deposition catalyst, a preparation method therefor and use thereof.

### BACKGROUND TECHNOLOGY

Chlorotrifluoroethylene is a colorless gas with a slight ether odor and good reactivity, which is an important fluorine-containing polymeric monomer, is also an important chemical intermediate, and has been widely used in pesticides, medicine, polymer materials and other fields.

A Chinese patent CN1110103604A discloses a catalyst for catalyzing hydrodechlorination, a preparation method therefor and use thereof. In the method, an alloy catalyst adopts the element Ru as a main body and selects any one or more of specified alloy elements, such as Re, Ti, Cr, Ni, Al, Co, Cu, Nb, Ta, Ru, Pt, or Ag, to form an alloy with the Ru; and an auxiliary agent is an alkali metal or a rare earth metal, and a carrier is an activated carbon carrier. When the catalyst is used in preparation of chlorotrifluoroethylene, the chlorotrifluoroethylene has a conversion rate of about 95.7% and a selectivity of 95.6%.

However, a key core problem in the synthesis of (chlorotrifluoroethylene) CTFE by a catalytic hydrogenation method is how to inhibit carbon deposition. Although the presence of carbon deposition does not affect the conversion rate of a raw material and the selectivity of a product in an initial stage of a reaction, a great impact is caused to the stability and service life of the catalyst. It is reported in existing documents that the carbon deposition is mainly affected by the acidity of an active site of the catalyst and the reaction temperature. Therefore, the carbon deposition can be effectively inhibited by reducing the acidity of the active site or increasing the catalytic activity (reducing the reaction temperature), thereby prolonging the service life of the catalyst.

A Chinese patent CN1589970A discloses a regeneration method for a catalyst for dehydrogenation of alkyl aromatics to produce alkenyl aromatics. In the method, water vapor and air are introduced to regenerate the catalyst by a hydrothermal method, but a high regeneration temperature is required in the method to completely burn carbon deposits on the catalyst.

A Chinese patent CN107497420A discloses a regeneration method for a carbon-containing precious metal catalyst. In the method, an oxygen content in a regenerated gas is controlled step by step in a combustion process to remove carbon deposits in the catalyst by combustion step by step, and then the activity of the catalyst is restored by chlorination and reduction. In a regeneration process of the precious metal catalyst, the activity of the catalyst will be reduced when a moisture content is too high in chlorination operation. Therefore, the moisture in a process gas and a maximum value of an operating temperature need to be strictly controlled in the method to ensure the regeneration efficiency.

A Chinese patent CN107999057A discloses a regeneration method for a supported precious metal catalyst. In the method, an inactivated supported precious metal catalyst is subjected to oxidation treatment with a mixed gas of CO₂ and O₂, followed by reduction with a reducing agent in a tetrahydrofuran solvent after the oxidation to obtain a regenerated catalyst.

In summary, existing catalysts and catalytic hydrogenation processes thereof still cannot completely inhibit the generation of carbon deposits, and the carbon deposits are removed only by a regeneration method after the generation of carbon deposits, that is, the carbon deposits are removed by adopting air, CO₂, H₂O and other gases to undergo chemical reactions with the carbon deposits. However, a carbon deposit removing process will inevitably damage carrier carbon, destroy particle structures of catalysts and cause irreversible inactivation of the catalysts. Therefore, the carbon deposit removing process is difficult to control.

Up to now, relevant reports on effective solutions of a carbon deposition problem of catalysts for catalyzing hydrodechlorination have not been found yet.

### SUMMARY OF THE INVENTION

The object of the present invention is to provide a catalyst capable of effectively resisting carbon deposition for hydrodechlorination of fluorochloroalkanes, a preparation method therefor and use thereof.

According to a first aspect of the present invention, the following technical schemes are adopted in the present invention.

An anti-carbon deposition catalyst, the catalyst is composed of a carbon carrier, a metal active component, a metal auxiliary agent I, and a metal auxiliary agent II. The metal active component is platinum or palladium, the metal auxiliary agent I is zinc or copper or cobalt, the metal auxiliary agent II is ruthenium or nickel, and each type of metal component includes one and only one metal.

The metal active component accounts for 0.2-2.0% of a mass content of the carrier, and a mass ratio of the metal active component, the metal auxiliary agent I and the metal auxiliary agent II is 1:(1-10):(0.01-0.001).

Preferably, the metal active component accounts for 0.2-1.5% of the mass content of the carrier.

Preferably, the mass ratio of the metal active component, the metal auxiliary agent I and the metal auxiliary agent II is 1:(1-8):(0.01-0.003).

The carbon carrier is activated carbon, preferably a granular type, with an ash content of less than 2 wt %.

According to a second aspect of the present invention, the present invention further provides a preparation method for an anti-carbon deposition catalyst. The preparation method specifically includes the following steps:

A1. activated carbon treatment step:
soaking activated carbon in a sodium hydroxide solution with a molar concentration of 1-5 mol/L at 50-90° C. for 2-6 hours and performing washing with water to neutral; and soaking the activated carbon in hydrochloric acid with a molar concentration of 0.5-3 mol/L at 20-60° C. for 2-6 hours, and performing washing with water to neutral; where a ratio of the activated carbon to the sodium hydroxide solution/hydrochloric acid is each 1:(1.5-3.0) (g/mL), and g/mL represents that 1 g of the activated carbon is soaked in the sodium hydroxide solution/hydrochloric acid per mL;
and the purpose of the step is to remove metal ash in the activated carbon to make a content of an individual metal component not higher than 0.01 wt %;

A2. formulation of an impregnating solution:
weighing a metal active component salt, a metal auxiliary agent I salt and a metal auxiliary agent II salt, and performing even mixing in an aqueous solution of ammonium citrate and hydroxyacetic acid to form an impregnating solution;
where all the metal active component salt, the metal auxiliary agent I salt and the metal auxiliary agent II salt are soluble salts; specifically, the metal active component salt may be a chloride or a nitrate of platinum or palladium, such as platinum dichloride, platinum tetrachloride, or palladium dichloride; the metal auxiliary agent I salt may be a soluble salt of zinc or copper or cobalt, for example, selected from a chloride, a nitrate, a sulfate, or an organic salt of the zinc or copper or cobalt, such as zinc chloride, copper chloride, cobalt chloride, copper nitrate, cobalt nitrate, zinc sulfate, copper sulfate, cobalt sulfate, zinc acetate, cobalt acetate, etc.; and the metal auxiliary agent II salt may be a soluble salt of ruthenium or nickel, such as hydrated ruthenium trichloride, ruthenium acetate, nickel chloride, nickel nitrate, nickel sulfate, nickel acetate, etc.;

A3. activated carbon impregnation:
adding the activated carbon treated in step A1 to the impregnating solution in step A2, performing stirring at 20-50° C. for 2-5 hours, then performing standing for aging for 5-12 hours, and removing and drying the activated carbon; where the drying may be air drying or oven drying; a volume of the impregnating solution is 1.5-2 times of a pore volume of the activated carbon, equivalent-volume impregnation is performed, and the pore volume of the activated carbon is measured by a BET method; and

A4. catalyst synthesis step:
calcining the activated carbon supported with metal components in an inert gas atmosphere from room temperature to 200-600° C. at a rate of 1-5° C./min, and maintaining a constant temperature for 2-5 hours to obtain an anti-carbon deposition catalyst. Specifically, the inert gas is nitrogen or argon at a flow rate of 1-10 mL/min.

By the calcining, not only the metal salts can be calcined to form oxides, but also the binding force between the metal components or between the metal components and the activated carbon carrier can be enhanced to improve the stability of the catalyst.

Further, a molar ratio of the ammonium citrate to the hydroxyacetic acid is 1:(1-3); and a molar ratio of a sum of the ammonium citrate and the hydroxyacetic acid to a total metal is 1:(1-3). Preferably, the molar ratio of the ammonium citrate to the hydroxyacetic acid is 1:(1.5-2.5); and the molar ratio of the sum of the ammonium citrate and the hydroxyacetic acid to the total metal is 1:(1.5-2.5).

According to a third aspect of the present invention, the present invention further provides use of the anti-carbon deposition catalyst prepared above. Specifically, the anti-carbon deposition catalyst is used in a hydrodechlorination reaction; and more specifically, the anti-carbon deposition catalyst is used in hydrodechlorination of trifluorotrichloroethane to prepare chlorotrifluoroethylene, hydrodechlorination of 1,1,2-trichloroethylene to prepare ethylene, hydrodechlorination of chloropentafluoroethane to prepare pentafluoroethane, hydrodechlorination of 1,1-dichlorotetrafluoroethane to prepare 1-chloro-tetrafluoroethane and tetrafluoroethane, or hydrodechlorination of 2,3-dichloro-1,1,1,4,4,4-hexafluoro-2-butene to prepare 1,1,1,4,4,4-hexafluoro-2-butene.

When the anti-carbon deposition catalyst of the present invention is used in the hydrodechlorination reaction, the anti-carbon deposition catalyst is subjected to reduction and activation before a feed gas is introduced to undergo the hydrodechlorination reaction, and the steps of reduction and activation include:
placing the anti-carbon deposition catalyst in a reactor, introducing hydrogen for reduction and activation at a hydrogen volume space velocity of 2-8 min⁻¹, performing a heating procedure from room temperature to 300-400° C. at 1-3° C./min, and maintaining a constant temperature for 1-3 hours.

Further, a ratio of a particle size of the catalyst to an inner diameter of the reactor is 1:(6-10).

Furthermore, the present application adopts that ammonia and the feed gas are simultaneously introduced into the reactor to undergo the hydrodechlorination reaction, a content of the ammonia is matched with that of generated hydrogen chloride, and a molar ratio of the two is configured at 1:1.

The present invention further provides a method for preparing chlorotrifluoroethylene by hydrodechlorination of trifluorotrichloroethane, which specifically includes:
simultaneously introducing ammonia, trifluorotrichloroethane (R113) and hydrogen into a tubular reactor to undergo a hydrodechlorination reaction at a reaction temperature of 250-350° C., where a space velocity of the trifluorotrichloroethane is 40-100 h⁻¹, a molar ratio of the R113 to the hydrogen is 1:(1-3), preferably 1:(1.5-2.5), and a content of the ammonia and generated hydrogen chloride are configured at 1:1.

In an actual reaction, a flow quantity of the ammonia is first configured according to a theoretical hydrogen chloride content obtained by the reaction with the R113 and the hydrogen. In a reaction process, the hydrogen chloride content in a product flow is monitored to adjust the flow quantity of the ammonia.

Compared with the prior art, advantages of the present invention are mainly embodied in the following points.

(1) Three metal auxiliary agents in the anti-carbon deposition catalyst of the present invention form a multifunctional catalytic activity center to achieve in-situ elimination of carbon deposits through hydrogenation while maintaining high dechlorination catalytic performance without causing macroscopic accumulation of carbon deposits, thereby effectively inhibiting the generation of carbon deposits and greatly improving the stability and service life of the catalyst.

(2) When the anti-carbon deposition catalyst of the present invention is used in the hydrodechlorination reaction, the alkaline ammonia is introduced while the feed gas is introduced, which can reduce the acidity of an active center, inhibit the adsorption of acidic hydrogen chloride and an adsorbed chloride on a surface of the catalyst and reduce the generation of carbon deposits. Meanwhile, an appropriate amount of the ammonia can also react with hydrogen chloride to promote the hydrodechlorination reaction to proceed to the right and increase the conversion rate.

### DESCRIPTION OF THE EMBODIMENTS

Embodiments listed in the present invention are described in detail by specific examples below, but the scope of protection of the present invention is not limited to the following examples.

A metal active component salt, a metal auxiliary agent I salt, a metal auxiliary agent II salt, sodium hydroxide, hydrochloric acid, ammonium citrate and hydroxyacetic acid used in the examples are all purchased from Sinopharm Chemical Reagent Co., Ltd., and activated carbon is purchased from Aladdin Chemical Procurement Platform. The activated carbon has a specific surface area of 1,100 m²/g, a pore volume of 0.7648 cc/g and an ash content of 1.5 wt %.

### Example 1

(1) 8.0 mg of PtCl₂, 8.0 mg of Cu(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:1, and a molar ratio of a sum of the ammonium citrate and the hydroxyacetic acid to a total metal was 1:2. Then, deionized water was added to prepare an impregnating solution with a total volume of 5.0 mL, and stirring was performed at 50° C. for 5 hours.

(2) 4 g of 10- to 20-mesh granular activated carbon was weighed, added to a beaker containing 6 mL of a 1 mol/L sodium hydroxide solution and stirred at 50° C. for 2 hours, and then the activated carbon was washed with deionized water to neutral. Then, the activated carbon was added to a beaker containing 6 mL of 0.5 mol/L hydrochloric acid and stirred at 20° C. for 2 hours, and then the activated carbon was washed with deionized water to neutral. Natural air drying was performed to prepare a treated activated carbon carrier.

(3) The treated granular activated carbon carrier was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 1 mL/min, and then the temperature was raised from room temperature to 220° C. at 1° C./min and maintained constant for 2 hours to obtain an anti-carbon deposition catalyst.

(4) The prepared catalyst was placed in a tubular reactor, where a ratio of a particle size of the catalyst to an inner diameter of the reactor was 1:6. Hydrogen was introduced for reduction and activation, where a volume space velocity of the hydrogen was 2 min⁻¹. Then, the temperature was raised from room temperature to 300° C. at a rate of 1° C./min and maintained constant for 3 hours.

(5) After the reduction and the activation were completed, the volume space velocity of the hydrogen was maintained at 2 min⁻¹, and vaporized trifluorotrichloroethane (R113) was introduced, where a volume space velocity of the R113 was 40 h⁻¹. Meanwhile, ammonia was introduced at a flow quantity of 1.4 min⁻¹, which was consistent with that of generated hydrogen chloride, and a reaction temperature was 250° C.

After stable operation for 10 hours, a chromatographic test was carried out. Area normalization results show that the R113 has a conversion rate of 96.45%, and chlorotrifluoroethylene has a selectivity of 96.78%.

### Example 2

(1) 8.0 mg of PtCl₂, 16.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:2, and a molar ratio of a sum of the ammonium citrate and the hydroxyacetic acid to a total metal was 1:1. Then, deionized water was added to prepare an impregnating solution with a total volume of 6.0 mL, and stirring was performed at 20° C. for 2 hours.

(2) 4 g of 10- to 20-mesh granular activated carbon was weighed, added to a beaker containing 6 mL of a 3 mol/L sodium hydroxide solution and stirred at 70° C. for 4 hours, and then the activated carbon was washed with deionized water to neutral. Then, the activated carbon was added to a beaker containing 6 mL of 1 mol/L hydrochloric acid and stirred at 40° C. for 4 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 3 mL/min, and then the temperature was raised from room temperature to 200° C. at 3° C./min and maintained constant for 3 hours to obtain an anti-carbon deposition catalyst.

(4) The prepared catalyst was placed in a tubular reactor, where a ratio of a particle size of the catalyst to an inner diameter of the reactor was 1:10. Hydrogen was introduced for reduction and activation, where a space velocity of the hydrogen was 8 min⁻¹. Then, the temperature was raised from room temperature to 400° C. at a rate of 3° C./min and maintained constant for 3 hours.

(5) After the reduction and the activation were completed, the space velocity of the hydrogen was maintained at 8 min⁻¹, and vaporized R113 was introduced, where a space velocity of the R113 was 100 h⁻¹. Meanwhile, ammonia was introduced at a flow quantity of 3.4 min⁻¹, which was consistent with that of generated hydrogen chloride, and a reaction temperature was 350° C.

After stable operation for 10 hours, a chromatographic test was carried out. Area normalization results show that the R113 has a conversion rate of 96.95%, and chlorotrifluoroethylene has a selectivity of 96.89%.

### Example 3

(1) 8.0 mg of PtCl₂, 40.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:3, and a molar ratio of a sum of the ammonium citrate and the hydroxyacetic acid to a total metal was 1:3. Then, deionized water was added to prepare an impregnating solution with a total volume of 6.5 mL, and stirring was performed at 30° C. for 3 hours.

(2) 4 g of 10- to 20-mesh granular activated carbon was weighed, added to a beaker containing 8 mL of a 5 mol/L sodium hydroxide solution and stirred at 90° C. for 6 hours, and then the activated carbon was washed with deionized water to neutral. Then, the activated carbon was added to a beaker containing 6 mL of 3 mol/L hydrochloric acid and stirred at 60° C. for 6 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 5 mL/min, and then the temperature was raised from room temperature to 200° C. at 5° C./min and maintained constant for 4 hours to obtain an anti-carbon deposition catalyst.

(4) The prepared catalyst was placed in a tubular reactor, where a ratio of a particle size of the catalyst to an inner diameter of the reactor was 1:8. Hydrogen was introduced for reduction and activation, where a space velocity of the hydrogen was 6 min⁻¹. Then, the temperature was raised from room temperature to 350° C. at 2° C./min and maintained constant for 2 hours.

(5) After the reduction and the activation were completed, the space velocity of the hydrogen was maintained at 6 min⁻¹, and vaporized R113 was introduced, where a space velocity of the R113 was 60 h⁻¹. Meanwhile, ammonia was introduced at a flow quantity of 2.1 min⁻¹, which was consistent with that of generated hydrogen chloride, and a reaction temperature was 280° C.

After stable operation for 10 hours, a chromatographic test was carried out. Area normalization results show that the R113 has a conversion rate of 97.23%, and chlorotrifluoroethylene has a selectivity of 97.18%.

### Example 4

(1) 12.0 mg of PtCl₂, 80.0 mg of Co(NO₃)₂ and 0.012 mg of nickel nitrate were weighed and poured into a beaker containing ammonium citrate and hydroxyacetic acid, where a molar ratio of the ammonium citrate to the hydroxyacetic acid was 1:3, and a molar ratio of a sum of the ammonium citrate and the hydroxyacetic acid to a total metal was 1:2. Then, deionized water was added to prepare an impregnating solution with a total volume of 7.0 mL, and stirring was performed at 50° C. for 2 hours.

(2) 4 g of 10- to 20-mesh granular activated carbon was weighed, added to a beaker containing 6 mL of a 4 mol/L sodium hydroxide solution and stirred at 80° C. for 5 hours, and then the activated carbon was washed with deionized water to neutral. Then, the activated carbon was added to a beaker containing 8 mL of 1 mol/L hydrochloric acid and stirred at 50° C. for 3 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 7 mL/min, and then the temperature was raised from room temperature to 250° C. at 5° C./min and maintained constant for 5 hours to obtain an anti-carbon deposition catalyst.

(4) The prepared catalyst was placed in a tubular reactor, where a ratio of a particle size of the catalyst to an inner diameter of the reactor was 1:9. Hydrogen was introduced for reduction and activation, where a space velocity of the hydrogen was 8 min⁻¹. Then, the temperature was raised from room temperature to 320° C. at a rate of 2° C./min and maintained constant for 2 hours.

(5) After the reduction and the activation were completed, the space velocity of the hydrogen was maintained at 8 min⁻¹, and vaporized R113 was introduced, where a volume space velocity of the R113 was 80 h⁻¹. Meanwhile, ammonia was introduced at a flow quantity of 2.7 min⁻¹, which was consistent with that of generated hydrogen chloride, and a reaction temperature was 300° C.

After stable operation for 10 hours, a chromatographic test was carried out. Area normalization results show that the R113 has a conversion rate of 97.65%, and chlorotrifluoroethylene has a selectivity of 97.09%.

### Example 5

(1) 16.0 mg of PtCl₂, 16.0 mg of Co(NO₃)₂ and 0.012 mg of nickel nitrate were weighed and poured into a beaker containing ammonium citrate and hydroxyacetic acid, where a molar ratio of the ammonium citrate to the hydroxyacetic acid was 1:2, and a molar ratio of a sum of the ammonium citrate and the hydroxyacetic acid to a total metal was 1:3. Then, deionized water was added to prepare an impregnating solution with a total volume of 6.0 mL, and stirring was performed at 20° C. for 5 hours.

(2) 4 g of 10- to 20-mesh granular activated carbon was weighed, added to a beaker containing 9 mL of a 1 mol/L sodium hydroxide solution and stirred at 50° C. for 2 hours, and then the activated carbon was washed with deionized water to neutral. Then, the activated carbon was added to a beaker containing 9 mL of 0.5 mol/L hydrochloric acid and stirred at 30° C. for 3 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 10 mL/min, and then the temperature was raised from room temperature to 250° C. at a rate of 5° C./min and maintained constant for 5 h to obtain an anti-carbon deposition catalyst.

(4) The prepared catalyst was placed in a tubular reactor, where a ratio of a particle size of the catalyst to an inner diameter of the reactor was 1:7. Hydrogen was introduced for reduction and activation, where a space velocity of the hydrogen was 4 min⁻¹. Then, the temperature was raised from room temperature to 400° C. at a rate of 1° C./min and maintained constant for 1 hour.

(5) After the reduction and the activation were completed, the space velocity of the hydrogen was maintained at 4 min⁻¹, and vaporized R113 was introduced, where a volume space velocity of the R113 was 70 h⁻¹. Meanwhile, ammonia was introduced at a flow quantity of 2.4 min⁻¹, which was consistent with that of generated hydrogen chloride, and a reaction temperature was 350° C.

After stable operation for 10 hours, a chromatographic test was carried out. Area normalization results show that the R113 has a conversion rate of 95.22%, and chlorotrifluoroethylene has a selectivity of 97.82%.

### Example 6

(1) 8.0 mg of PtCl₂, 16.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:1, and a molar ratio of a sum of the ammonium citrate and the hydroxyacetic acid to a total metal was 1:1. Then, deionized water was added to prepare an impregnating solution with a total volume of 7.0 mL, and stirring was performed at 50° C. for 3 hours.

(2) 4 g of 10- to 20-mesh granular activated carbon was weighed, added to a beaker containing 9 mL of a 1 mol/L sodium hydroxide solution and stirred at 50° C. for 4 hours, and then the activated carbon was washed with deionized water to neutral. Then, the activated carbon was added to a beaker containing 6 mL of 0.5 mol/L hydrochloric acid and stirred at 50° C. for 4 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 10 mL/min, and then the temperature was raised from room temperature to 220° C. at a rate of 3° C./min and maintained constant for 5 hours to obtain an anti-carbon deposition catalyst.

(4) The prepared catalyst was placed in a tubular reactor, where a ratio of a particle size of the catalyst to an inner diameter of the reactor was 1:10. Hydrogen was introduced for reduction and activation, where a space velocity of the hydrogen was 5 min⁻¹. Then, the temperature was raised from room temperature to 400° C. at a rate of 3° C./min and maintained constant for 3 hours.

(5) After the reduction and the activation were completed, the space velocity of the hydrogen was maintained at 5 min⁻¹, and vaporized R113 was introduced, where a space velocity of the R113 was 100 h⁻¹. Meanwhile, ammonia was introduced at a flow quantity of 3.4 min⁻¹, which was consistent with that of generated hydrogen chloride, and a reaction temperature was 330° C.

After stable operation for 10 hours, a chromatographic test was carried out. Area normalization results show that the R113 has a conversion rate of 96.65%, and chlorotrifluoroethylene has a selectivity of 96.21%.

### Example 7

(1) 8.0 mg of PtCl₂, 16.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:3, and a molar ratio of a sum of the ammonium citrate and the hydroxyacetic acid to a total metal was 1:1. Then, deionized water was added to prepare an impregnating solution with a total volume of 5.5 mL, and stirring was performed at 30° C. for 2 hours.

(2) 4 g of 10- to 20-mesh granular activated carbon was weighed, added to a beaker containing 6 mL of a 3 mol/L sodium hydroxide solution and stirred at 80° C. for 6 hours, and then the activated carbon was washed with deionized water to neutral. Then, the activated carbon was added to a beaker containing 6 mL of 3 mol/L hydrochloric acid and stirred at 50° C. for 4 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 1 mL/min, and then the temperature was raised from room temperature to 200° C. at a rate of 1° C./min and maintained constant for 2 hours to obtain an anti-carbon deposition catalyst.

(4) The prepared catalyst was placed in a tubular reactor, where a ratio of a particle size of the catalyst to an inner diameter of the reactor was 1:9. Hydrogen was introduced for reduction and activation, where a space velocity of the hydrogen was 8 min⁻¹. Then, the temperature was raised from room temperature to 300° C. at a rate of 3° C./min and maintained constant for 1 hour.

(5) After the reduction and the activation were completed, the space velocity of the hydrogen was maintained at 8 min⁻¹, and vaporized R113 was introduced, where a space velocity of the R113 was 90 h⁻¹. Meanwhile, ammonia was introduced at a flow quantity of 3.1 min⁻¹, which was consistent with that of generated hydrogen chloride, and a reaction temperature was 330° C.

After stable operation for 10 hours, a chromatographic test was carried out. Area normalization results show that the R113 has a conversion rate of 96.45%, and chlorotrifluoroethylene has a selectivity of 97.34%.

### Example 8

(1) 8.0 mg of PdCl₂, 80.0 mg of Cu(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:2, and a molar ratio of a sum of the ammonium citrate and the hydroxyacetic acid to a total metal was 1:1. Then, deionized water was added to prepare an impregnating solution with a total volume of 6.0 mL, and stirring was performed at 30° C. for 5 hours.

(2) 4 g of 10- to 20-mesh granular activated carbon was weighed, added to a beaker containing 10 mL of a 1 mol/L sodium hydroxide solution and stirred at 50° C. for 2 hours, and then the activated carbon was washed with deionized water to neutral. Then, the activated carbon was added to a beaker containing 10 mL of 0.5 mol/L hydrochloric acid and stirred at 20° C. for 2 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 10 mL/min, and then the temperature was raised from room temperature to 250° C. at a rate of 1° C./min and maintained constant for 3 hours to obtain an anti-carbon deposition catalyst.

(4) The prepared catalyst was placed in a tubular reactor, where a ratio of a particle size of the catalyst to an inner diameter of the reactor was 1:8. Hydrogen was introduced for reduction and activation, where a space velocity of the hydrogen was 8 min⁻¹. Then, the temperature was raised from room temperature to 380° C. at a rate of 1° C./min and maintained constant for 3 hours.

(5) After the reduction and the activation were completed, the space velocity of the hydrogen was maintained at 8 min⁻¹, and vaporized R113 was introduced, where a space velocity of the R113 was 60 h⁻¹. Meanwhile, ammonia was introduced at a flow quantity of 2.1 min⁻¹, which was consistent with that of generated hydrogen chloride, and a reaction temperature was 350° C.

After stable operation for 10 hours, a chromatographic test was carried out. Area normalization results show that the R113 has a conversion rate of 98.75%, and chlorotrifluoroethylene has a selectivity of 95.87%.

### Example 9

(1) 8.0 mg of PtCl₂, 16.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:2, and a molar ratio of a sum of the ammonium citrate and the hydroxyacetic acid to a total metal was 1:2. Then, deionized water was added to prepare an impregnating solution with a total volume of 7.0 mL, and stirring was performed at 30° C. for 3 hours.

(2) 4 g of 10- to 20-mesh granular activated carbon was weighed, added to a beaker containing 12 mL of a 1 mol/L sodium hydroxide solution and stirred at 60° C. for 4 hours, and then the activated carbon was washed with deionized water to neutral. Then, the activated carbon was added to a beaker containing 6 mL of 0.5 mol/L hydrochloric acid and stirred at 30° C. for 4 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 10 mL/min, and then the temperature was raised from room temperature to 250° C. at a rate of 1° C./min and maintained constant for 5 h to obtain an anti-carbon deposition catalyst.

(4) The prepared catalyst was placed in a tubular reactor, where a ratio of a particle size of the catalyst to an inner diameter of the reactor was 1:10. Hydrogen was introduced for reduction and activation, where a space velocity of the hydrogen was 8 min⁻¹. Then, the temperature was raised from room temperature to 400° C. at a rate of 2° C./min and maintained constant for 3 hours.

(5) After the reduction and the activation were completed, the space velocity of the hydrogen was maintained at 8 min⁻¹, and vaporized R113 was introduced, where a space velocity of the R113 was 100 h⁻¹. Meanwhile, ammonia was introduced at a flow quantity of 3.4 min⁻¹, which was consistent with that of generated hydrogen chloride, and a reaction temperature was 340° C.

After stable operation for 10 hours, a chromatographic test was carried out. Area normalization results show that the R113 has a conversion rate of 96.35%, and chlorotrifluoroethylene has a selectivity of 96.77%.

### Example 10

(1) 8.0 mg of PtCl₂, 80.0 mg of Cu(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:3, and a molar ratio of a sum of the ammonium citrate and the hydroxyacetic acid to a total metal was 1:2.5. Then, deionized water was added to prepare an impregnating solution with a total volume of 5.5 mL, and stirring was performed at 50° C. for 5 hours.

(2) 4 g of 10- to 20-mesh granular activated carbon was weighed, added to a beaker containing 12 mL of a 1 mol/L sodium hydroxide solution and stirred at 90° C. for 6 hours, and then the activated carbon was washed with deionized water to neutral. Then, the activated carbon was added to a beaker containing 12 mL of 3 mol/L hydrochloric acid and stirred at 60° C. for 6 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 8 mL/min, and then the temperature was raised from room temperature to 250° C. at a rate of 4° C./min and maintained constant for 3 hours to obtain an anti-carbon deposition catalyst.

(4) The prepared catalyst was placed in a tubular reactor, where a ratio of a particle size of the catalyst to an inner diameter of the reactor was 1:10. Hydrogen was introduced for reduction and activation, where a space velocity of the hydrogen was 5 min⁻¹. Then, the temperature was raised from room temperature to 370° C. at a rate of 3° C./min and maintained constant for 2 hours.

(5) After the reduction and the activation were completed, the space velocity of the hydrogen was maintained at 5 min⁻¹, and vaporized trifluorotrichloroethane (R113) was introduced, where a volume space velocity of the R113 was 80 h⁻¹. Meanwhile, ammonia was introduced at a flow quantity of 2.7 min⁻¹, which was consistent with that of generated hydrogen chloride, and a reaction temperature was 270° C.

After stable operation for 10 hours, a chromatographic test was carried out. Area normalization results show that the R113 has a conversion rate of 97.85%, and chlorotrifluoroethylene has a selectivity of 95.86%.

### Example 11

(1) 8.0 mg of PdCl₂, 80.0 mg of Zn(NO₃)₂ and 0.08 mg of ruthenium chloride were weighed and poured into a beaker containing ammonium citrate and hydroxyacetic acid, where a molar ratio of the ammonium citrate to the hydroxyacetic acid was 1:1.5, and a molar ratio of a sum of the ammonium citrate and the hydroxyacetic acid to a total metal was 1:3. Then, deionized water was added to prepare an impregnating solution with a total volume of 6.0 mL, and stirring was performed at 50° C. for 5 hours.

(2) 4 g of 10- to 20-mesh granular activated carbon was weighed, added to a beaker containing 10 mL of a 5 mol/L sodium hydroxide solution and stirred at 80° C. for 3 hours, and then the activated carbon was washed with deionized water to neutral. Then, the activated carbon was added to a beaker containing 12 mL of 2 mol/L hydrochloric acid and stirred at 50° C. for 2 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 5 mL/min, and then the temperature was raised from room temperature to 250° C. at a rate of 3° C./min and maintained constant for 3 hours to obtain an anti-carbon deposition catalyst.

(4) The prepared catalyst was placed in a tubular reactor, where a ratio of a particle size of the catalyst to an inner diameter of the reactor was 1:7. Hydrogen was introduced for reduction and activation, where a space velocity of the hydrogen was 4 min⁻¹. Then, the temperature was raised from room temperature to 340° C. at a rate of 2° C./min and maintained constant for 2 hours.

(5) After the reduction and the activation were completed, the space velocity of the hydrogen was maintained at 4 min⁻¹, and vaporized R113 was introduced, where a space velocity of the R113 was 90 h⁻¹. Meanwhile, ammonia was introduced at a flow quantity of 3.1 min⁻¹, which was consistent with that of generated hydrogen chloride, and a reaction temperature was 260° C.

After stable operation for 10 hours, a chromatographic test was carried out. Area normalization results show that the R113 has a conversion rate of 97.15%, and chlorotrifluoroethylene has a selectivity of 96.45%.

### Example 12

(1) 8.0 mg of PdCl₂, 80.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:2, and a molar ratio of a sum of the ammonium citrate and the hydroxyacetic acid to a total metal was 1:2.5. Then, deionized water was added to prepare an impregnating solution with a total volume of 5.5 mL, and stirring was performed at 50° C. for 5 hours.

(2) 4 g of 10- to 20-mesh granular activated carbon was weighed, added to a beaker containing 10 mL of a 1 mol/L sodium hydroxide solution and stirred at 50° C. for 5 hours, and then the activated carbon was washed with deionized water to neutral. Then, the activated carbon was added to a beaker containing 12 mL of 0.5 mol/L hydrochloric acid and stirred at 20° C. for 6 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 10 mL/min, and then the temperature was raised from room temperature to 230° C. at a rate of 5° C./min and maintained constant for 4 h to obtain an anti-carbon deposition catalyst.

(4) The prepared catalyst was placed in a tubular reactor, where a ratio of a particle size of the catalyst to an inner diameter of the reactor was 1:7. Hydrogen was introduced for reduction and activation, where a space velocity of the hydrogen was 6 min⁻¹. Then, the temperature was raised from room temperature to 330° C. at a rate of 3° C./min and maintained constant for 1 hour.

(5) After the reduction and the activation were completed, the space velocity of the hydrogen was maintained at 6 min⁻¹, and vaporized R113 was introduced, where a space velocity of the R113 was 100 h⁻¹. Meanwhile, ammonia was introduced at a flow quantity of 3.4 min⁻¹, which was consistent with that of generated hydrogen chloride, and a reaction temperature was 300° C.

After stable operation for 10 hours, a chromatographic test was carried out. Area normalization results show that the R113 has a conversion rate of 97.05%, and chlorotrifluoroethylene has a selectivity of 97.09%.

### Example 13

(1) 8.0 mg of PtCl₂, 16.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:3, and a molar ratio of a sum of the ammonium citrate and the hydroxyacetic acid to a total metal was 1:2. Then, deionized water was added to prepare an impregnating solution with a total volume of 6.0 mL, and stirring was performed at 50° C. for 5 hours.

(2) 4 g of 10- to 20-mesh granular activated carbon was weighed, added to a beaker containing 9 mL of a 1 mol/L sodium hydroxide solution and stirred at 90° C. for 2 hours, and then the activated carbon was washed with deionized water to neutral. Then, the activated carbon was added to a beaker containing 9 mL of 0.5 mol/L hydrochloric acid and stirred at 60° C. for 2 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 10 mL/min, and then the temperature was raised from room temperature to 250° C. at a rate of 1° C./min and maintained constant for 5 h to obtain an anti-carbon deposition catalyst.

(4) The prepared catalyst was placed in a tubular reactor, where a ratio of a particle size of the catalyst to an inner diameter of the reactor was 1:7. Hydrogen was introduced for reduction and activation, where a space velocity of the hydrogen was 5 min⁻¹. Then, the temperature was raised from room temperature to 400° C. at a rate of 1° C./min and maintained constant for 3 hours.

(5) After the reduction and the activation were completed, the space velocity of the hydrogen was maintained at 5 min⁻¹, and vaporized R113 was introduced, where a space velocity of the R113 was 60 h⁻¹. Meanwhile, ammonia was introduced at a flow quantity of 2.1 min⁻¹, which was consistent with that of generated hydrogen chloride, and a reaction temperature was 320° C.

After stable operation for 10 hours, a chromatographic test was carried out. Area normalization results show that the R113 has a conversion rate of 97.93%, and chlorotrifluoroethylene has a selectivity of 96.59%.

### Example 14

(1) 8.0 mg of PtCl₂, 80.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:3, and a molar ratio of a sum of the ammonium citrate and the hydroxyacetic acid to a total metal was 1:2.5. Then, deionized water was added to prepare an impregnating solution with a total volume of 5.0 mL, and stirring was performed at 50° C. for 5 hours.

(2) 4 g of 10- to 20-mesh granular activated carbon was weighed, added to a beaker containing 7 mL of a 1 mol/L sodium hydroxide solution and stirred at 50° C. for 5 hours, and then the activated carbon was washed with deionized water to neutral. Then, the activated carbon was added to a beaker containing 8 mL of 0.5 mol/L hydrochloric acid and stirred at 20° C. for 5 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 8 mL/min, and then the temperature was raised from room temperature to 250° C. at a rate of 5° C./min and maintained constant for 2 h to obtain an anti-carbon deposition catalyst.

(4) The prepared catalyst was placed in a tubular reactor, where a ratio of a particle size of the catalyst to an inner diameter of the reactor was 1:10. Hydrogen was introduced for reduction and activation, where a space velocity of the hydrogen was 6 min⁻¹. Then, the temperature was raised from room temperature to 320° C. at a rate of 1° C./min and maintained constant for 3 hours.

(5) After the reduction and the activation were completed, the space velocity of the hydrogen was maintained at 6 min⁻¹, and a vaporized feed gas was introduced, where a space velocity of the feed gas was 50 h⁻¹. Meanwhile, ammonia was introduced at a flow quantity of 1.7 min⁻¹, which was consistent with that of generated hydrogen chloride, and a reaction temperature was 260° C.

After stable operation for 10 hours, a chromatographic test was carried out. Area normalization results show that the R113 has a conversion rate of 96.90%, and chlorotrifluoroethylene has a selectivity of 96.69%.

### Example 15

(1) 8.0 mg of PtCl₂, 16.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:1, and a molar ratio of a sum of the ammonium citrate and the hydroxyacetic acid to a total metal was 1:2. Then, deionized water was added to prepare an impregnating solution with a total volume of 6.5 mL, and stirring was performed at 50° C. for 5 hours.

(2) 4 g of 10- to 20-mesh granular activated carbon was weighed, added to a beaker containing 6 mL of a 1 mol/L sodium hydroxide solution and stirred at 50° C. for 2 hours, and then the activated carbon was washed with deionized water to neutral. Then, the activated carbon was added to a beaker containing 9 mL of 3 mol/L hydrochloric acid and stirred at 20° C. for 5 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 1 mL/min, and then the temperature was raised from room temperature to 250° C. at a rate of 1° C./min and maintained constant for 2 hours to obtain an anti-carbon deposition catalyst.

(4) The prepared catalyst was placed in a tubular reactor, where a ratio of a particle size of the catalyst to an inner diameter of the reactor was 1:10. Hydrogen was introduced for reduction and activation, where a space velocity of the hydrogen was 3 min⁻¹. Then, the temperature was raised from room temperature to 380° C. at a rate of 1° C./min and maintained constant for 1 hour.

(5) After the reduction and the activation were completed, the space velocity of the hydrogen was maintained at 3 min⁻¹, and a vaporized feed gas was introduced, where a space velocity of the feed gas was 50 h⁻¹. Meanwhile, ammonia was introduced at a flow quantity of 2.7 min⁻¹, which was consistent with that of generated hydrogen chloride, and a reaction temperature was 310° C.

After stable operation for 10 hours, a chromatographic test was carried out. Area normalization results show that the R113 has a conversion rate of 96.65%, and chlorotrifluoroethylene has a selectivity of 96.45%.

### Example 16

(1) 8.0 mg of PtCl₂, 16.0 mg of Co(NO₃)₂ and 0.08 mg of nickel nitrate were weighed and poured into a beaker containing ammonium citrate and hydroxyacetic acid, where a molar ratio of the ammonium citrate to the hydroxyacetic acid was 1:2, and a molar ratio of a sum of the ammonium citrate and the hydroxyacetic acid to a total metal was 1:3. Then, deionized water was added to prepare an impregnating solution with a total volume of 6.0 mL, and stirring was performed at 50° C. for 5 hours.

(2) 4 g of 10- to 20-mesh granular activated carbon was weighed, added to a beaker containing 6 mL of a 1 mol/L sodium hydroxide solution and stirred at 50° C. for 6 hours, and then the activated carbon was washed with deionized water to neutral. Then, the activated carbon was added to a beaker containing 9 mL of 3 mol/L hydrochloric acid and stirred at 20° C. for 5 hours, and then the activated carbon was washed with deionized water to neutral. Natural air drying was performed to prepare a treated activated carbon carrier.

(3) The treated granular activated carbon carrier in step (2) was poured into the impregnating solution prepared in step (1) for impregnation in a slightly stirred state of the impregnating solution, and after the impregnation was completed, standing overnight. Drying was performed in a nitrogen atmosphere at a nitrogen flow rate of 1 mL/min, and then the temperature was raised from room temperature to 250° C. at a rate of 5° C./min and maintained constant for 4 h to obtain an anti-carbon deposition catalyst.

(4) The prepared catalyst was placed in a tubular reactor, where a ratio of a particle size of the catalyst to an inner diameter of the reactor was 1:10. Hydrogen was introduced for reduction and activation, where a space velocity of the hydrogen was 6 min⁻¹. Then, the temperature was raised from room temperature to 400° C. at a rate of 2° C./min and maintained constant for 2 hours.

(5) After the reduction and the activation were completed, the space velocity of the hydrogen was maintained at 6 min⁻¹, and a vaporized feed gas was introduced, where a space velocity of the feed gas was 80 h⁻¹. Meanwhile, ammonia was introduced at a flow quantity of 2.7 min⁻¹, which was consistent with that of generated hydrogen chloride, and a reaction temperature was 330° C.

After stable operation for 10 hours, a chromatographic test was carried out. Area normalization results show that the R113 has a conversion rate of 96.85%, and chlorotrifluoroethylene has a selectivity of 96.08%.

### Example 17

Operation in the present example was the same as that in Example 2, only the reaction temperature and the space velocity of the feed gas R113 in Example 2 were changed, and catalyst performance under different reaction conditions is compared. Results are shown in Table 1 below.

TABLE 1
Catalytic performance under different
reaction conditions in Example 2

| Reaction | | Conversion | |
|---|---|---|---|
| temperature/° C. | Space velocity/h⁻¹ | rate/% | Selectivity/% |
| 250 | 40 | 96.59 | 96.78 |
| 270 | 50 | 96.89 | 96.48 |
| 290 | 60 | 97.18 | 96.39 |
| 310 | 70 | 97.88 | 97.01 |
| 330 | 80 | 98.04 | 96.89 |
| 350 | 90 | 98.78 | 96.78 |
| 330 | 100 | 97.45 | 96.88 |

Note:
A ratio of a particle size of a catalyst to an inner diameter of a reactor was 1:10, a volume space velocity of hydrogen was 8 min⁻¹, and then the temperature was raised from room temperature to 400° C. at a rate of 3° C./min and maintained constant for 3 h. After reduction and activation were completed, the space velocity of the hydrogen was maintained, and vaporized R113 was introduced, where a flow quantity of ammonia was set consistent with that of generated hydrogen chloride.

### Example 18

In the present example, a service life experiment in Example 2 was tested, that is, reactants in Example 2 were detected and analyzed at different stable operation times. Results are shown in Table 2 below.

TABLE 2
Service life experiment in Example 2

| Time/h | Conversion rate/% | Selectivity/% |
|---|---|---|
| 10 | 96.95 | 96.89 |
| 20 | 97.25 | 95.99 |
| 30 | 97.19 | 96.48 |
| 40 | 96.45 | 96.89 |
| 50 | 96.87 | 96.12 |
| 60 | 96.58 | 96.59 |
| 70 | 97.12 | 96.49 |
| 80 | 97.38 | 96.87 |
| 90 | 97.89 | 96.29 |
| 100 | 97.99 | 96.37 |
| 110 | 96.89 | 96.98 |
| 120 | 96.48 | 96.46 |
| 130 | 96.67 | 96.59 |
| 140 | 96.19 | 97.01 |
| 150 | 96.87 | 97.12 |
| 160 | 96.58 | 97.23 |
| 170 | 96.78 | 96.87 |
| 180 | 97.15 | 96.49 |
| 190 | 97.03 | 96.67 |
| 200 | 97.45 | 96.82 |

Test method: A ratio of a particle size of a catalyst to an inner diameter of a reactor was 1:10, a space velocity of hydrogen was 8 min⁻¹, and then the temperature was raised from room temperature to 400° C. at a rate of 3° C./min and maintained constant for 3 h. After reduction and activation were completed, the space velocity of the hydrogen was maintained at 8 min⁻¹, and vaporized R113 was introduced, where a volume space velocity of the R113 was 100 h⁻¹, and a flow quantity of ammonia was 3.4 min⁻¹. A reaction temperature was 350° C.

### Comparative Example 1

The present comparative example was compared with Example 2 to demonstrate the importance of the metal active component for the catalyst performance. A preparation method was the same as that in Example 2, but only had the difference that the metal auxiliary agent II, namely nickel, was not added.

TABLE 3
Catalytic performance results in Comparative Example 1

| Time | Conversion rate % | Selectivity % |
|---|---|---|
| 10 | 96.25 | 96.32 |
| 20 | 96.65 | 96.58 |
| 30 | 96.19 | 96.48 |
| 40 | 96.45 | 96.29 |
| 50 | 96.55 | 96.57 |
| 60 | 96.38 | 96.46 |
| 70 | 96.02 | 96.28 |
| 80 | 96.38 | 96.58 |
| 90 | 96.29 | 96.89 |
| 100 | 96.01 | 95.48 |
| 110 | 95.89 | 96.78 |
| 120 | 94.48 | 95.67 |
| 130 | 93.67 | 96.24 |
| 140 | 92.19 | 95.13 |
| 150 | 91.02 | 95.78 |
| 160 | 90.68 | 96.48 |
| 170 | 90.02 | 96.78 |
| 180 | 87.28 | 96.49 |
| 190 | 84.61 | 95.88 |
| 200 | 81.26 | 96.01 |

Test method: A ratio of a particle size of a catalyst to an inner diameter of a reactor was 1:10, a space velocity of hydrogen was 8 min⁻¹, and then the temperature was raised from room temperature to 400° C. at a rate of 3° C./min and maintained constant for 3 hours. After reduction and activation were completed, the space velocity of the hydrogen was maintained at 8 min-1, and vaporized R113 was introduced, where a volume space velocity of the R113 was 100 h⁻¹, and a flow quantity of ammonia was 3.4 min⁻¹. A reaction temperature was 350° C.

### Comparative Example 2

The present comparative example was compared with Example 2 to demonstrate the importance of a loading method of the metal active component for the catalyst performance. A preparation method was the same as that in Example 2, but only had the difference that the impregnating solution was formulated by not adding the ammonium citrate and the hydroxyacetic acid.

TABLE 4
Catalytic performance results in Comparative Example 2

| Time | Conversion rate % | Selectivity % |
|---|---|---|
| 10 | 94.25 | 95.45 |
| 20 | 94.65 | 95.28 |
| 30 | 94.12 | 94.78 |
| 40 | 93.85 | 95.89 |
| 50 | 93.95 | 95.58 |
| 60 | 94.38 | 96.14 |
| 70 | 94.08 | 95.89 |
| 80 | 94.78 | 96.23 |
| 90 | 94.69 | 97.01 |
| 100 | 94.01 | 97.06 |
| 110 | 94.00 | 96.54 |
| 120 | 93.28 | 96.48 |
| 130 | 92.67 | 95.14 |
| 140 | 92.12 | 95.14 |
| 150 | 91.09 | 95.18 |
| 160 | 90.68 | 95.68 |
| 170 | 89.02 | 95.45 |
| 180 | 86.18 | 95.23 |
| 190 | 85.21 | 95.02 |
| 200 | 83.46 | 95.58 |

### Comparative Example 3

The present comparative example was compared with Example 2 to demonstrate the importance of the ammonia for the catalyst performance. A preparation method was the same as that in Example 2, but only had the difference that the ammonia was not introduced in performance testing.

TABLE 5
Catalytic performance results in Comparative Example 3

| Time | Conversion rate % | Selectivity % |
|---|---|---|
| 10 | 97.15 | 96.47 |
| 20 | 97.56 | 96.49 |
| 30 | 97.35 | 96.56 |
| 40 | 97.75 | 96.49 |
| 50 | 97.25 | 96.53 |
| 60 | 96.88 | 96.75 |
| 70 | 96.92 | 95.78 |
| 80 | 96.94 | 96.28 |
| 90 | 97.29 | 96.65 |
| 100 | 96.91 | 95.78 |
| 110 | 96.95 | 96.57 |
| 120 | 97.02 | 95.89 |
| 130 | 96.67 | 95.94 |
| 140 | 96.09 | 95.83 |
| 150 | 95.02 | 96.74 |
| 160 | 94.68 | 96.48 |
| 170 | 93.02 | 96.67 |
| 180 | 91.56 | 96.30 |
| 190 | 90.45 | 95.48 |
| 200 | 88.86 | 96.41 |

### Comparative Example 4

The present comparative example was compared with Example 2 to demonstrate the importance of the metal auxiliary agent II and the ammonia for the catalyst performance. A preparation method was the same as that in Example 2, but only had the differences that the metal auxiliary agent II, namely nickel, was not added, and the ammonia was not introduced.

TABLE 6
Catalytic performance results in Comparative Example 4

| Time | Conversion rate % | Selectivity % |
|---|---|---|
| 10 | 95.29 | 95.24 |
| 20 | 95.64 | 95.26 |
| 30 | 95.23 | 95.14 |
| 40 | 95.85 | 95.35 |
| 50 | 95.55 | 95.65 |
| 60 | 95.31 | 95.48 |
| 70 | 95.41 | 95.38 |
| 80 | 94.98 | 95.78 |
| 90 | 94.79 | 95.48 |
| 100 | 94.09 | 94.68 |
| 110 | 94.49 | 96.01 |
| 120 | 93.48 | 95.48 |
| 130 | 91.27 | 95.69 |
| 140 | 89.31 | 95.89 |
| 150 | 87.12 | 96.02 |
| 160 | 85.68 | 95.78 |
| 170 | 82.22 | 95.48 |
| 180 | 79.14 | 95.67 |
| 190 | 78.11 | 95.64 |
| 200 | 75.26 | 95.20 |

### Comparative Example 5

The present comparative example was compared with Example 2 to demonstrate the importance of a loading method of the metal active component for the catalyst performance. A preparation method was the same as that in Example 2, but only had the difference that the impregnating solution was formulated by only adding the ammonium citrate and not adding the hydroxyacetic acid.

TABLE 7
Catalytic performance results in Comparative Example 5

| Time/h | Conversion rate/% | Selectivity/% |
|---|---|---|
| 10 | 95.12 | 96.81 |
| 20 | 95.30 | 96.93 |
| 30 | 95.28 | 97.12 |
| 40 | 94.97 | 96.57 |
| 50 | 95.02 | 97.16 |
| 60 | 95.19 | 96.56 |
| 70 | 95.14 | 96.64 |
| 80 | 95.31 | 97.21 |
| 90 | 95.10 | 96.77 |
| 100 | 95.27 | 96.62 |
| 110 | 95.30 | 96.98 |
| 120 | 95.48 | 97.17 |
| 130 | 95.67 | 96.80 |
| 140 | 95.19 | 97.11 |
| 150 | 94.97 | 96.72 |
| 160 | 93.77 | 96.93 |
| 170 | 92.78 | 96.84 |
| 180 | 89.15 | 96.56 |
| 190 | 87.03 | 97.02 |
| 200 | 86.45 | 96.89 |

### Comparative Example 6

The present comparative example was compared with Example 2 to demonstrate the importance of a loading method of the metal active component for the catalyst performance. A preparation method was the same as that in Example 2, but only had the difference that the impregnating solution was formulated by only adding the hydroxyacetic acid and not adding the ammonium citrate.

TABLE 8
Catalytic performance results in Comparative Example 6

| Time/h | Conversion rate/% | Selectivity/% |
|---|---|---|
| 10 | 95.22 | 96.97 |
| 20 | 95.31 | 97.00 |
| 30 | 95.25 | 96.96 |
| 40 | 95.17 | 97.04 |
| 50 | 95.26 | 97.10 |
| 60 | 95.25 | 96.94 |
| 70 | 95.36 | 97.05 |
| 80 | 95.29 | 97.01 |
| 90 | 95.11 | 96.29 |
| 100 | 95.23 | 97.03 |
| 110 | 95.27 | 96.80 |
| 120 | 95.38 | 96.79 |
| 130 | 95.20 | 96.62 |
| 140 | 94.35 | 96.82 |
| 150 | 93.23 | 97.07 |
| 160 | 92.00 | 96.73 |
| 170 | 91.21 | 96.81 |
| 180 | 88.73 | 97.10 |
| 190 | 85.29 | 96.91 |
| 200 | 83.30 | 96.66 |

1. An anti-carbon deposition catalyst comprising a carbon carrier, a metal active component, a metal auxiliary agent I, and a metal auxiliary agent II, wherein the metal active component is platinum or palladium, the metal auxiliary agent I is zinc or copper or cobalt, the metal auxiliary agent II is ruthenium or nickel, and each type of metal component comprises one and only one metal; and the metal active component accounts for 0.2-2.0% of a mass content of the carrier, and a mass ratio of the metal active component, the metal auxiliary agent I and the metal auxiliary agent II is 1:(1-10):(0.01-0.001).

2. The anti-carbon deposition catalyst according to claim 1, wherein the metal active component accounts for 0.2-1.5% of the mass content of the carrier.

3. The anti-carbon deposition catalyst according to claim 1 or 2, wherein the mass ratio of the metal active component, the metal auxiliary agent I and the metal auxiliary agent II is 1:(1-8):(0.01-0.003).

4. A preparation method for an anti-carbon deposition catalyst, comprising the following steps:
A1. activated carbon treatment:
soaking activated carbon in a sodium hydroxide solution with a molar concentration of 1-5 mol/L at 50-90° C. for 2-6 hours, and performing washing with water to neutral; and soaking the activated carbon in hydrochloric acid with a molar concentration of 0.5-3 mol/L at 20-60° C. for 2-6 hours, and performing washing with water to neutral; wherein a ratio of the activated carbon to the sodium hydroxide solution/hydrochloric acid is each 1:(1.5-5.0) (g/mL);
A2. formulation of an impregnating solution:
weighing a metal active component salt, a metal auxiliary agent I salt and a metal auxiliary agent II salt, and performing even mixing in an aqueous solution of ammonium citrate and hydroxyacetic acid to form an impregnating solution;
A3. activated carbon impregnation:
adding the activated carbon treated in step A1 to the impregnating solution in step A2, performing stirring at 20-50° C. for 2-5 hours, then performing standing for aging for 5-12 hours, and removing and drying the activated carbon; wherein a volume of the impregnating solution is 1.5-2 times of a pore volume of the activated carbon; and
A4. catalyst synthesis step:
calcining the activated carbon supported with metal components in an inert gas atmosphere from room temperature to 200-600° C. at a rate of 1-5° C./min, and maintaining a constant temperature for 2-5 h to obtain an anti-carbon deposition catalyst.

5. The preparation method for an anti-carbon deposition catalyst according to claim 4, wherein a molar ratio of the ammonium citrate to the hydroxyacetic acid is 1:(1-3); a molar ratio of a sum of the ammonium citrate and the hydroxyacetic acid to a total metal is 1:(1-3); and the inert gas is nitrogen or argon at a flow rate of 1-10 mL/min.

6. A method of using the anti-carbon deposition catalyst according to claim 1 in a hydrodechlorination reaction, wherein the anti-carbon deposition catalyst is used in hydrodechlorination of trifluorotrichloroethane to prepare chlorotrifluoroethylene, hydrodechlorination of 1,1,2-trichloroethylene to prepare ethylene, hydrodechlorination of chloropentafluoroethane to prepare pentafluoroethane, hydrodechlorination of 1,1-dichlorotetrafluoroethane to prepare 1-chloro-tetrafluoroethane and tetrafluoroethane, or hydrodechlorination of 2,3-dichloro-1,1,1,4,4,4-hexafluoro-2-butene to prepare 1,1,1,4,4,4-hexafluoro-2-butene.

7. The method of the anti-carbon deposition catalyst in a hydrodechlorination reaction according to claim 6, wherein the anti-carbon deposition catalyst is subjected to reduction and activation before a feed gas is introduced to undergo the hydrodechlorination reaction, and the steps of reduction and activation comprise:
placing the anti-carbon deposition catalyst in a reactor, introducing hydrogen for reduction and activation at a hydrogen volume space velocity of 2-8 min⁻¹, performing a heating procedure from room temperature to 300-400° C. at 1-3° C./min, and maintaining a constant temperature for 1-3 hours.

8. The method of the anti-carbon deposition catalyst in a hydrodechlorination reaction according to claim 7, wherein a ratio of a particle size of the catalyst to an inner diameter of the reactor is 1:(6-10).

9. The method of the anti-carbon deposition catalyst in a hydrodechlorination reaction according to claim 8, wherein ammonia and the feed gas are simultaneously introduced into the reactor to undergo the hydrodechlorination reaction, a content of the ammonia is matched with that of generated hydrogen chloride, and a molar ratio of the two is configured at 1:1.

10. A method for preparing chlorotrifluoroethylene by hydrodechlorination of trifluorotrichloroethane, wherein ammonia, trifluorotrichloroethane and hydrogen are simultaneously introduced into a tubular reactor to undergo a hydrodechlorination reaction at a reaction temperature of 250-350° C., a space velocity of the trifluorotrichloroethane is 40-100 h⁻¹, a molar ratio of the trifluorotrichloroethane to the hydrogen is 1:(1-3), and a content of the ammonia and generated hydrogen chloride are configured at 1:1.
